(19) United States
(12) Patent Application Publication
(10) Pub. No.: US 2026/0285867 A1
(43) Pub. Date: Sep. 24, 2026

(54) IMIDAZOTRIAZINE DERIVATIVES AS IL-17 MODULATORS

(71) Applicant: UCB BIOPHARMA SRL, Brussels (BE)

(72) Inventors: Jeffrey BRUFFAERTS, Slough, Berkshire (GB); Gregory William HASLETT, Slough, Berkshire (GB)

(21) Appl. No.: 19/475,094

(22) PCT Filed: Apr. 15, 2024
(86) PCT No.: PCT/EP2024/060136

§ 371 Date: Oct. 14, 2025

(30) Foreign Application Priority Data
Apr. 17, 2023 (GB) 2305587.4

(51) Int. Cl. C07D 487/04; A61K 31/53; A61P 29/00; A61P 37/02

(52) U.S. Cl. CPC C07D 487/04; A61K 31/53; A61P 29/00; A61P 37/02

(57) ABSTRACT

N—[(S)-(4,4-Difluorocyclohexyl){3-[1-(2,2-difluoropropylcarbamoyl)-3-hydroxy-3-methylcyclobutyl]imidazo[1,2-b][1,2,4]triazin-6-yl}methyl]-4-methyl-1,2,5-oxadiazole-3-carboxamide, or a pharmaceutically acceptable salt thereof, being potent modulators of human IL-17 activity, are accordingly of benefit in the treatment and/or prevention of various human ailments, including inflammatory and autoimmune disorders.

The present invention relates to heterocyclic compounds, and to their use in therapy. More particularly, this invention is concerned with pharmacologically active substituted imidazo[1,2-b][1,2,4]triazine derivatives. These compounds act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J. Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Korn et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2023/275301, and also WO 2020/261141, describe fused bicyclic imidazole derivatives that are stated to act as modulators of IL-17 activity, and thus to be of benefit in the treatment of pathological conditions including adverse inflammatory and autoimmune disorders.

None of the prior art available to date, however, discloses or suggests the precise structural class of substituted imidazo[1,2-b][1,2,4]triazine derivatives as provided by the present invention.

As well as being potent modulators of human IL-17 activity, the compounds in accordance with the present invention possess other notable advantages. In particular, the compounds of the invention display valuable metabolic stability, as determined in either microsomal or hepatocyte incubations. The compounds of the invention also display valuable permeability as determined by standard assays, e.g. the Caco-2 permeability assay.

The present invention provides a compound of formula (I):

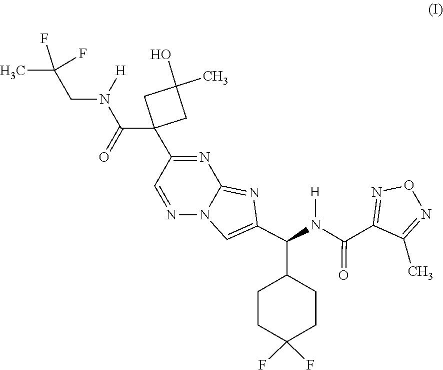 [SMILES: Cc1nonc1C(=O)N[C@H](c1cn2ncc(C3(C(=O)NCC(C)(F)F)CC(C)(O)C3)nc2n1)C1CCC(F)(F)CC1]

or a pharmaceutically acceptable salt thereof.

The compounds in accordance with the present invention are encompassed within the generic scope of WO 2023/275301. There is, however, no specific disclosure therein of a compound of formula (I) as defined above, or a pharmaceutically acceptable salt thereof.

The present invention also provides a compound of formula (I) as defined above, or a pharmaceutically acceptable salt thereof, for use in therapy.

The present invention also provides a compound of formula (I) as defined above, or a pharmaceutically acceptable salt thereof, for use in the treatment and/or prevention of disorders for which the administration of a modulator of IL-17 function is indicated.

The present invention also provides the use of a compound of formula (I) as defined above, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above, or a pharmaceutically acceptable salt thereof.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

Formula (I) and the formulae depicted hereinafter are intended to represent all individual stereoisomers and all possible mixtures thereof, unless stated or shown otherwise. In addition, compounds of formula (I) may exist as tautomers, for example amide (NHC═O)→hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium, D) or ³H (tritium, T) atom, preferably ¹H. Similarly, by way of example, each individual carbon atom present in formula (I), or in the formulae depicted hereinafter, may be present as a ¹²C, ¹³C or ¹⁴C atom, preferably ¹²C.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, axial spondyloarthritis, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, hidradenitis suppurativa, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydria and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, hidradenitis suppurativa, scleroderma, systemic sclerosis, lung fibrosis, inflammatory bowel diseases (including Crohn's disease and ulcerative colitis), axial spondyloarthritis, ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis, hidradenitis suppurativa, axial spondyloarthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compound of formula (I) above may be prepared by a process which comprises reacting a compound of formula (II) with the compound of formula (III):

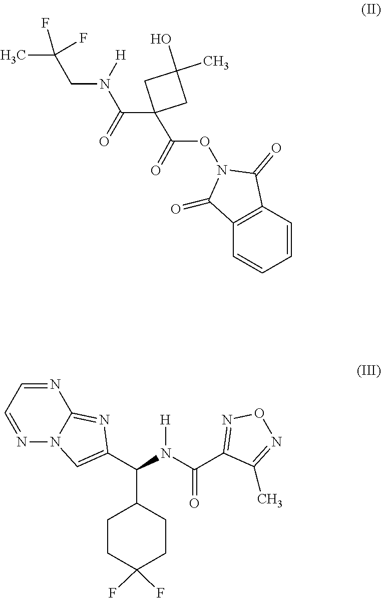 [SMILES: CC(F)(F)CNC(=O)C1(C(=O)ON2C(=O)c3ccccc3C2=O)CC(C)(O)C1 ; Cc1nonc1C(=O)N[C@H](c1cn2nccnc2n1)C1CCC(F)(F)CC1]

in the presence of a transition metal catalyst.

Suitable transition metal catalysts of use in the reaction include [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate; and tris[2-phenylpyridinato-C²,N]iridium(III). The reaction will generally be performed by exposing the reactants to a bright light source. A suitable bright light source will typically comprise the ‘integrated photoreactor’ described in ACS Cent. Sci., 2017, 3, 647-653; or the Penn Photoreactor M2 or M1 system; or the Pennoc system. The reaction will conveniently be carried out at ambient temperature, optionally in the presence of trifluoroacetic acid, and in a suitable solvent, e.g. a dipolar aprotic solvent such as N,N-dimethylformamide, or an organic sulfoxide such as dimethyl sulfoxide.

The intermediate of formula (III) above may be prepared by the procedure described in WO 2023/275301, or by methods analogous thereto.

The intermediates of formula (II) above may be prepared by a two-step process which comprises:
(i) saponifying a compound of formula (IV):

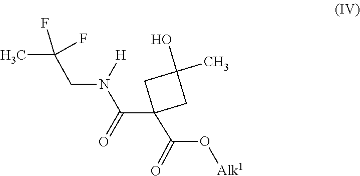

wherein Alk¹ represents C₁₋₄ alkyl, e.g. methyl; and
(ii) reaction of the carboxylic acid derivative thereby obtained with N-hydroxyphthalimide.

Where Alk¹ represents methyl, the saponification reaction in step (i) will generally be effected by treatment with a base. Suitable bases include inorganic hydroxides, e.g. an alkali metal hydroxide such as lithium hydroxide or sodium hydroxide. The reaction is conveniently performed at ambient or elevated temperature in water and a suitable organic solvent, e.g. a cyclic ether such as tetrahydrofuran, or a C₁₋₄ alkanol such as methanol or ethanol, or a chlorinated solvent such as dichloromethane.

Step (ii) may conveniently be accomplished in the presence of a coupling agent such as N-(3-dimethylaminopropyl)-N′-ethylcarbodiimide hydrochloride. The reaction is suitably performed at ambient temperature, optionally in the presence of 4-(dimethyl-amino)pyridine, and in a suitable solvent, e.g. a dipolar aprotic solvent such as N,N-dimethylformamide.

The intermediates of formula (IV) above may be prepared by reacting a carboxylic acid derivative of formula (V):

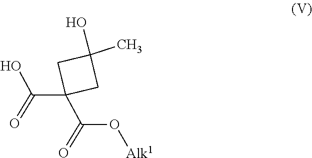

wherein Alk¹ is as defined above; with 2,2-difluoropropylamine or a salt thereof, e.g. the hydrochloride salt.

The reaction is conveniently accomplished in the presence of a coupling agent and a base. Suitable coupling agents include 1-[bis(dimethylamino)methylene]-1H-1,2,3-triazolo[4,5-b]pyridinium 3-oxid hexafluorophosphate (HATU); and 2,4,6-tripropyl-1,3,5,2,4,6-trioxatriphosphorinane-2,4,6-trioxide; and 2-chloro-1-methylpyridinium iodide. Suitable bases include organic amines, e.g. a trialkylamine such as N,N-diisopropylethylamine; or pyridine. The reaction is conveniently performed at ambient or elevated temperature in a suitable solvent, e.g. a cyclic ether such as tetrahydrofuran; or a dipolar aprotic solvent such as N,N-dimethylformamide or N,N-dimethylacetamide; or a chlorinated solvent such as dichloromethane; or an organic ester solvent such as ethyl acetate.

Where they are not commercially available, the starting materials of formula (V) may be prepared by methods analogous to those described in the accompanying Examples, or by standard methods well known from the art.

Where a mixture of products is obtained from any of the processes described above for the preparation of a compound according to the invention, the desired product can be separated therefrom at an appropriate stage by conventional methods such as preparative HPLC; or column chromatography utilising, for example, silica and/or alumina in conjunction with an appropriate solvent system.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit IL-17 induced IL-6 release from human dermal fibroblasts. Thus, when tested in the HDF cell line assay described below, the compounds of the present invention generally exhibit a pIC₅₀ value in excess of 7.5 (pIC₅₀ equals −log₁₀[IC₅₀], in which IC₅₀ is expressed as a molar concentration, so the skilled person will appreciate that a higher pIC₅₀ figure denotes a more active compound).

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 μL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

10 μL from the aqueous dilution plate was transferred to the reagent plate containing 30 μL of the diluted cytokines, to give a 2.5% DMSO solution. The compounds were incubated with the cytokine mixtures for 5 h at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 μL), then supernatant (10 μL) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm).

When tested in the HDF cell line assay as described above, the compounds of the accompanying Examples were found to exhibit the following pIC₅₀ values.

| Example | pIC₅₀ |
|---|---|
| 1 (Peak 1) | 7.7 |
| 2 (Peak 2) | 8.4 |

### Comparative Data

By way of comparison, the title compounds of Examples 17 and 56 of WO 2023/275301 are stated therein to exhibit pIC₅₀ values of 7.9 and 7.8 respectively.

The following Examples illustrate the preparation of compounds according to the invention.

### EXAMPLES

### Abbreviations

MeOH: methanol THF: tetrahydrofuran
EtOAc: ethyl acetate DMSO: dimethyl sulfoxide
DIPEA: N,N-diisopropylethylamine DMF: N,N-dimethylformamide
TFA: trifluoroacetic acid LDA: lithium diisopropylamide
EDCI·HCl: N-(3-dimethylaminopropyl)-N′-ethylcarbodiimide hydrochloride HATU: 1-[bis(dimethylamino)methylene]-1H-1,2,3-triazolo[4,5-b]pyridinium 3-oxid hexafluorophosphate
{Ir[dF(CF₃)ppy]₂(dtbpy)}PF₆: [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate
h: hour r.t.: room temperature
M: mass; molar RT: retention time
HPLC: High Performance Liquid Chromatography
LCMS: Liquid Chromatography Mass Spectrometry
SFC: Supercritical Fluid Chromatography
TOF-MS: Time-of-Flight Mass Spectrometry

### Analytical and Separation Methods

### Method 1

Stationary Phase: Phenomenex Gemini NX-C18 2×20 mm, 3 μm
Column Temperature: 40° C.
Mobile Phase A: 10 mM ammonium formate in water+0.1% ammonia solution
Mobile Phase B: acetonitrile+5% water+0.1% ammonia solution
Flow rate: 1 mL/minute
Gradient program: Time A % B %
   0.00 95.00 5.00
   1.50 5.00 95.00
   2.25 5.00 95.00
   2.50 95.00 5.00

### Method 2

Stationary Phase: Phenomenex Gemini NX-C18 2×50 mm, 3 μm
Column Temperature: 40° C.
Mobile Phase A: 10 mM ammonium formate in water+0.1% formic acid
Mobile Phase B: acetonitrile+5% water+0.1% formic acid
Flow rate: 1 mL/minute
Gradient program: Time A % B %
   0.00 95.00 5.00
   1.80 5.00 95.00
   2.10 5.00 95.00
   2.30 95.00 5.00

### Method 3

Stationary Phase: Waters Acquity UPLC BEH C18 2.1×50 mm, 1.7 μm
Mobile Phase A: 10 mM ammonium formate in water+0.1% ammonia solution
Mobile Phase B: acetonitrile+5% water+0.1% ammonia solution
Flow rate: 1.5 mL/minute
Gradient program: Time A % B %
   0.00 95.00 5.00
   0.10 95.00 5.00
   3.50 5.00 95.00
   4.00 5.00 95.00
   4.05 95.00 5.00

### Method 4

Chiral analysis performed using a Lux Cellulose-4, 150×4.6 mm, 3 μm column, flow rate 3 mL/minute, column temperature 35° C., eluting with a 3-40% MeOH (+0.1% NH₄OH) method ABPR 120 bar), using a 6.5 minute run time on a Waters UPC² Acquity system, in tandem with a Waters QDa mass spectrometer.

### Intermediate 1

### Methyl 3-hydroxy-3-methyleyclobutanecarboxylate

A solution of cis-3-hydroxy-3-methylcyclobutanecarboxylic acid (97 mass %) (850 mg, 6.33 mmol) in MeOH (100 mass %) (7.5 mL, 190 mmol) and H₂SO₄ (17.82 mol/L, 1.5 mL, 27 mmol) was stirred at 70° C. After 2 h, the reaction mixture was diluted with water (25 mL), and EtOAc (50 mL) was added. The phases were separated. The organic phase was washed with saturated aqueous Na₂CO₃ solution (2×10 mL), then concentrated in vacuo, to yield the title compound (770 mg, 84.3%) as a colourless liquid. δ_{H} (300 MHz, CDCl₃) 4.03 (s, 1H), 3.49 (s, 3H), 2.52-2.41 (m, 1H), 2.20-2.01 (m, 4H), 1.19 (s, 3H).

### Intermediate 2

### 3-Hydroxy-1-methoxycarbonyl-3-methylcyclobutanecarboxylic Acid

To a solution of Intermediate 1 (100 mass %) (600 mg, 4.1618 mmol) in THF (20 ml), in a three-neck 100 mL flask at −78° C. under N₂, was added LDA in hexanes (1.8 mol/L, 6 mL, 11 mmol) over 5 minutes. After 30 minutes, CO₂ gas (generated from dry ice) was bubbled through the reaction mixture for 20 minutes, maintaining the internal temperature at −78° C., then the reaction mixture was allowed to warm to r.t. over 20 minutes. After a further 20 minutes of bubbling CO₂ through the solution, the reaction was quenched with water (2 mL) and aqueous NaOH solution (1M, 20 mL), then EtOAc (10 mL) was added. The phases were separated. The aqueous phase was adjusted to pH ~1 with concentrated HCl (approximately 3 mL), then EtOAc (40 mL) was added. The phases were separated, and the aqueous phase was washed with EtOAc (4×10 mL). The combined organic fractions were concentrated in vacuo to yield the title compound (450 mg, 57%) as a colourless oil. δ_{H} (300 MHz, CDCl₃) 6.97 (br s, 2H), 3.79 (s, 3H), 2.74-2.70 (m, 4H), 1.37-1.40 (m, 3H).

### Intermediate 3

### Methyl 1-(2,2-difluoropropylcarbamoyl)-3-hydroxy-3-methylcyclobutanecarboxylate

To a solution of Intermediate 2 (1935 mg, 10.28 mmol) and DIPEA (9 mL, 51.41 mmol) in DMF (21 mL, 265 mmol) was added HATU (5100 mg, 13.37 mmol) at r.t. After 5 minutes, 2,2-difluoropropylamine hydrochloride (2250 mg, 16.45 mmol) was added. The reaction mixture was stirred overnight, then diluted with brine (150 mL) and EtOAc (50 mL). The phases were separated, and the aqueous phase was washed with EtOAc (3×15 mL). The combined organic fractions were concentrated in vacuo. The residue was purified by normal phase chromatography (100 g silica, eluting with a gradient of 0-80% EtOAc/isohexane) to obtain, after concentration, the title compound (~1:1 mixture of isomers) (2050 mg, 75%) as a pale yellow oil. LCMS (Method 1): [M+H]⁺ 266.0, RT 0.40 minutes.

### Intermediate 4

### 1-(2,2-Difluoropropylcarbamoyl)-3-hydroxy-3-methylcyclobutanecarboxylic Acid

A solution of Intermediate 3 (2050 mg, 7.73 mmol), LiOH·H₂O (1100 mg, 46.37 mmol), MeOH (7.7 mL), THF (7.7 mL) and water (7.7 mL) was stirred vigorously at r.t. for 1.5 h, then concentrated in vacuo. To the crude residue was added water (20 mL), and the material was adjusted to pH ~1 with concentrated HCl (approximately 4 mL). EtOAc (50 mL) was added and the phases were separated. The aqueous phase was washed with EtOAc (4×5 mL), and the combined organic fractions were concentrated, to afford the title compound (~1:1 mixture of isomers) as a white foamy solid. δ_{H} (400 MHz, DMSO-d₆) 12.53 (s, 1H), 8.20 (t, J 6.4 Hz, 0.5H), 8.04 (t, J 6.3 Hz, 0.5H), 5.02 (s, 1H), 3.52 (m, 2H), 2.47 (m, 4H), 1.53 (m, 3H), 1.15-0.97 (m, 3H).

### Intermediate 5

### (1,3-Dioxoisoindolin-2-yl) 1-(2,2-difluoropropylcarbamoyl)-3-hydroxy-3-methylcyclobutanecarboxylate

To a solution of Intermediate 4 (1780 mg, 7.08 mmol) and N-hydroxyphthalimide (1450 mg, 8.86 mmol) in DMF (20 mL, 262 mmol) was added EDCI·HCl (1700 mg, 8.86 mmol). The reaction mixture was stirred vigorously at r.t. for 20 h, then diluted with brine (150 mL) and EtOAc (50 mL). The phases were separated, and the aqueous phase was washed with EtOAc (2×10 mL). The combined organic fractions were concentrated in vacuo. The residue was purified by normal phase chromatography (100 g silica, eluting with a gradient of 0-65% EtOAc/isohexane) to yield the title compound (~1:1 mixture of isomers) (1750 mg, 58%) as a colourless gel. LCMS (Method 2): [M+H]⁺ 397.0, RT 0.80 minutes.

### Examples 1 & 2

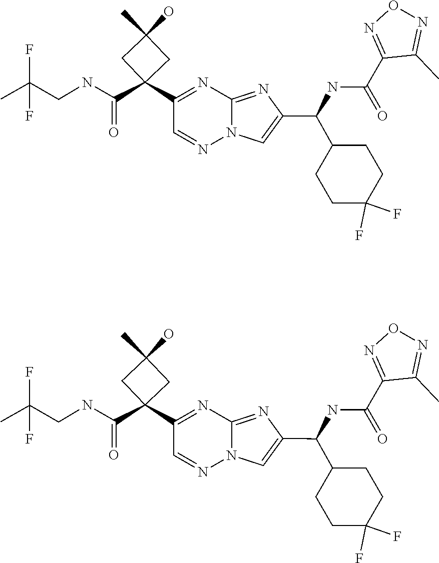

### N—[(S)-(4,4-Difluorocyclohexyl){3-[1-(2,2-difluoropropylcarbamoyl)-3-hydroxy-3-methylcyclobutyl]imidazo[1,2-b][1,2,4]triazin-6-yl}methyl]-4-methyl-1,2,5-oxadiazole-3-carboxamide (Syn and Anti Stereoisomers)

In a 40 mL vial, a solution of N—[(S)-(4,4-difluorocyclohexyl)(imidazo[1,2-b]-[1,2,4]triazin-6-yl)methyl]-4-methyl-1,2,5-oxadiazole-3-carboxamide (WO 2023/275301, Intermediate 21) (500 mg, 1.32 mmol), Intermediate 5 (788 mg, 1.99 mmol), TFA (0.15 mL, 1.99 mmol) and {Ir[dF(CF₃)ppy]₂(dtbpy)}PF₆ (30 mg, 0.026 mmol) in DMSO (27 mL, 370 mmol) was purged with N₂ for 1 minute, then stirred under photoirradiation (450 nm, Pennoc system, 1000 stirring rpm, 2500 fan stirring rpm). After 60 h, the reaction mixture was diluted with brine (100 mL), saturated aqueous NaHCO₃ solution (50 mL) and EtOAc (25 mL). The phases were separated, and the aqueous phase was washed with EtOAc (3×10 mL). The combined organic fractions were concentrated in vacuo. The residue was purified by chiral SFC to yield the title compounds (Peak 1, 76 mg, 10%; and Peak 2, 48 mg, 6%) as pale yellow solids.

Peak 1 (methyl assigned as being syn to the amide): δ_{H} (400 MHz, DMSO-d₆) 9.50 (d, J 8.8 Hz, 1H), 8.61 (s, 1H), 8.29 (s, 1H), 8.25 (t, J 6.3 Hz, 1H), 5.21 (t, J 8.5 Hz, 1H), 5.03 (s, 1H), 3.51 (td, J 13.9, 6.3 Hz, 2H), 2.91-2.79 (m, 2H), 2.74 (d, J 13.1 Hz, 2H), 2.48 (s, 3H), 2.36-1.47 (m, 9H), 1.51 (t, J 19.0 Hz, 3H), 1.27 (s, 3H). ¹³C NMR (101 MHz, DMSO-d₆) δ 173.11, 157.36, 156.06, 152.05, 149.85, 147.61, 140.86, 138.44, 127.02, 125.75, 124.64, 123.37, 122.25, 120.98, 112.96, 67.51, 52.46, 46.65, 46.04, 44.85, 44.55, 44.25, 33.21, 33.09, 32.97, 32.85, 32.74, 32.62, 28.51, 26.20, 26.11, 25.53, 25.44, 22.04, 21.78, 21.52, 8.99. ¹⁹F NMR (282 MHz, DMSO-d₆) δ −90.18 (d, J 232.5 Hz, 1F), −93.82 (m, 2F), −99.45 (d, J 232.4 Hz, 1F). LCMS (Method 3): [M+H]⁺ 583.2, RT 1.67 minutes. TOF-MS (positive m/z): [M+H]⁺ calculated for C₂₅H₃₁N₈O₄F₄: 583.2404. found: 583.2404. Chiral analysis (Method 4): RT 2.52 minutes.

Peak 2 (OH assigned as being syn to the amide): δ_{H} (400 MHz, DMSO-d₆) 9.51 (d, J 8.9 Hz, 1H), 8.61 (s, 1H), 8.30 (s, 1H), 8.23 (t, J 6.3 Hz, 1H), 5.22 (t, J 8.6 Hz, 2H), 3.50 (td, J 13.8, 6.3 Hz, 2H), 2.87-2.73 (m, 4H), 2.48 (s, 3H), 2.35-1.25 (m, 9H), 1.50 (t, J 19.1 Hz, 3H), 1.08 (s, 3H). ³C NMR (101 MHz, DMSO-d₆) δ 173.73, 157.35, 155.36, 152.06, 149.85, 147.51, 140.83, 139.10, 127.04, 125.84, 124.66, 123.45, 122.27, 121.07, 113.03, 67.23, 52.41, 45.94, 45.70, 44.79, 44.48, 44.18, 33.20, 33.08, 32.96, 32.85, 32.73, 32.61, 28.86, 26.27, 26.18, 25.52, 25.43, 21.92, 21.66, 21.40, 8.99. ¹⁹F NMR (282 MHz, DMSO-d₆) δ −90.17 (d, J 232.4 Hz, 1F), −93.57 (qt, J 19.1 Hz, 13.8 Hz, 2F), −99.44 (d, J 232.5 Hz, 1F). LCMS (Method 3): [M+H]⁺ 583.2, RT 1.71 minutes. TOF-MS (positive m/z): [M+H]⁺ calculated for C₂₅H₃₁N₈O₄F₄: 583.2404. found: 583.2391. Chiral analysis (Method 4): RT 2.78 minutes.

1. A compound of formula (I): 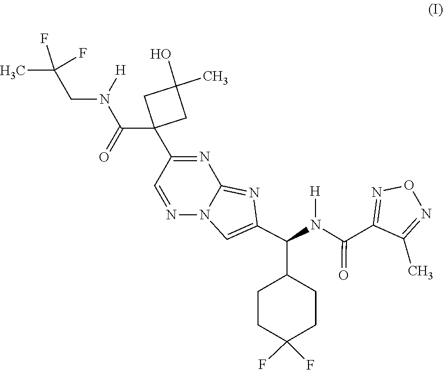 [SMILES: Cc1nonc1C(=O)N[C@H](c1cn2ncc(C3(C(=O)NCC(C)(F)F)CC(C)(O)C3)nc2n1)C1CCC(F)(F)CC1]
or a pharmaceutically acceptable salt thereof.

2. (canceled)

3. (canceled)

4. (canceled)

5. A pharmaceutical composition comprising a compound of formula (I) as depicted in claim 1, or a pharmaceutically acceptable salt thereof, in association with a pharmaceutically acceptable carrier.

6. (canceled)

7. (canceled)

8. A method for the treatment and/or prevention of disorders for which the administration of a modulator of IL-17 function is indicated, which comprises administering to a patient in need of such treatment an effective amount of a compound of formula (I) as depicted in claim 1, or a pharmaceutically acceptable salt thereof.

9. A method for the treatment and/or prevention of an inflammatory or autoimmune disorder, which comprises administering to a patient in need of such treatment an effective amount of a compound of formula (I) as depicted in claim 1, or a pharmaceutically acceptable salt thereof.
